SEQUENCE LISTING (36 sequences)

SEQ ID NO: 1 - DNA, 18 bp, Artificial Sequence
  Target site of p2Luc-U6A-0
tcgacgaattttttatgg

SEQ ID NO: 2 - DNA, 21 bp, Artificial Sequence
  Target site of p2Luc-U6A
tcgacgaattttttagggtgg

SEQ ID NO: 3 - DNA, 24 bp, Artificial Sequence
  Target site of p2Luc-U6A -6
tcgacgaattttttagggcagtgg

SEQ ID NO: 4 - DNA, 27 bp, Artificial Sequence
  Target site of p2Luc-U6A -9
tcgacgaattttttagggcagagctgg

SEQ ID NO: 5 - DNA, 21 bp, Artificial sequence
  Target site of p2Luc-U6A AT
tcgaattttttagggatataa

SEQ ID NO: 6 - DNA, 21 bp, Artificial sequence
  Target site of p2Luc-U6A GC
tcgaattttttaggggcgggc

SEQ ID NO: 7 - DNA, 18 bp, Artificial sequence
  Target site of p2Luc-A6C
tcgtcaaaaaacttgtgg

SEQ ID NO: 8 - DNA, 18 bp, Artificial sequence
  Target site of p2Luc-A6G
tcgtcaaaaaagttgtgg

SEQ ID NO: 9 - DNA, 18 bp, Artificial sequence
  target site of p2Luc-A6U
tcgtcaaaaaatttgtgg

SEQ ID NO: 10 - DNA, 18 bp, Artificial sequence
  Target site of p2Luc-UUUAAAC
tcgcctttaaaccagtgg

SEQ ID NO: 11 - DNA, 18 bp, Artificial sequence
  Target site of p2Luc-GGGAAAC
tcgcagggaaacggatgg

SEQ ID NO: 12 - DNA, 18 bp, Artificial sequence
  Target site of p2Luc-AAAUUUA
tcgacaaatttatagtgg

SEQ ID NO: 13 - DNA, 25 bp, Artificial sequence
  Control MOA
atccttcaacttccctgagctcgaa

SEQ ID NO: 14 - DNA, 25 bp, Artificial sequence
  MOA-1
cagggaagttgaaggatcccaccct

SEQ ID NO: 15 - DNA, 25 bp, Artificial sequence
  MOAA
ctcagggaagttgaaggatcccacc

SEQ ID NO: 16 - DNA, 25 bp, Artificial sequence
  MOAB
agctcagggaagttgaaggatccca

SEQ ID NO: 17 - DNA, 25 bp, Artificial sequence
  MOAC
cgagctcagggaagttgaaggatcc

SEQ ID NO: 18 - DNA, 25 bp, Artificial sequence
  MOAD
ttcgagctcagggaagttgaaggat

SEQ ID NO: 19 - DNA, 25 bp, Artificial sequence
  MOAE
tcttcgagctcagggaagttgaagg

SEQ ID NO: 20 - DNA, 25 bp, Artificial sequence
  MOA dmm3
acctcagcgaagttgaagcatccca

SEQ ID NO: 21 - DNA, 25 bp, Artificial sequence
  MOA dmm4
acctcagcgaagttgaagcatcgca

SEQ ID NO: 22 - DNA, 25 bp, Artificial sequence
  MOA dmm5
acctcagcgaacttgaagcatcgca

SEQ ID NO: 23 - DNA, 25 bp, Artificial sequence
  MOA AT
tcagggaagttgaaggatcttatat

SEQ ID NO: 24 - DNA, 25 bp, Artificial sequence
  MOA GC
tcagggaagttgaaggatcgcccgc

SEQ ID NO: 25 - DNA, 22 bp, Artificial sequence
  Target site of p2luc-U6A-0
tcgacgaattttttatgggatc

SEQ ID NO: 26 - DNA, 27 bp, Artificial
  Target site of p2Luc+1
tcgacgtgctcctgatgcccctggatc

SEQ ID NO: 27 - RNA, 20 nt, Artificial Sequence
  AZ1A
aguugaaggauccaggggca

SEQ ID NO: 28 - RNA, 20 nt, Artificial sequence
  AZ1B
ggaaguugaaggauccaggg

SEQ ID NO: 29 - RNA, 20 nt, Artificial Sequence
  AZ1C
cagggaaguugaaggaucca

SEQ ID NO: 30 - DNA, 12 bp, Artificial sequence
  Target site of p2Luc-Rare
tcgcgagaatgg

SEQ ID NO: 31 - DNA, 55 bp, Mus musculus
gcaaagttctttgaaagagcaataaaatggcttcaactatctgagtgacactgtg

SEQ ID NO: 32 - RNA, 27 nt, Artificial sequence
  2′-O-methyl antisense oligonucleotide to promote frameshift at target site in dystrophin gene of mdx mouse
cacagugucacucagauaguucaagcc

SEQ ID NO: 33 - RNA, 79 nt, Homo sapiens
guggccgucgaggaccgagucaggcagcugcaugaagcccacagggacuuugguccagcaucucagcacuuucuuucca

SEQ ID NO: 34 - RNA, 25 nt, Artificial sequence
  2′-O-methyl antisense oligonucleotide to promote frameshift at target site in mutation id number DMD_e53e60.
gggcuucaugcagcugccugacucg

SEQ ID NO: 35 - RNA, 214 nt, Homo sapiens
gggacgaacagggaggauccguguccugucuuuuaaaacuggcaucauuucccuguaccuuuucaagcaaguggcaaguucaacaggauuuugugaccagcgcaggcugggccuccuucugcaugauucuauccaaauuccaagacaguugggugaaguugcauccuuugggggcaguaacauugagccaaguguccggagcugcuuccaauuu

SEQ ID NO: 36 - RNA, 25 nt, Artificial sequence
  2′-O-methyl antisense oligonucleotide to promote frameshift at target site in mutation id number DMD_e64e65.
uugaaaagguacagggaaaugaugc

(19) United States
(12) Patent Application Publication
(10) Pub. No.: US 2011/0046200 A1
(43) Pub. Date: Feb. 24, 2011

(54) Use of antisense oligonucleotides to effect translation modulation

(76) Inventors: Michael T Howard, Salt Lake City, UT (US); John T. Atkins, Salt Lake City, UT (US)

Correspondence Address: Workman Nydegger;1000 Eagle Gate Tower, 60 East South Temple, Salt Lake City, UT, 84111, US

(21) Appl. No.: 11/659,452

(22) PCT Filed: Aug. 3, 2005
(86) PCT No.: PCT/US05/27455

§ 371 Date: Jun. 29, 2010

Related U.S. Application Data
(60) Provisional application No. 60/599,089, filed on Aug. 3, 2004.

(51) Int. Cl. A61K 31/7088; A61P 3/06; A61P 3/10; A61P 1/00; A61P 21/00; C12N 5/02; C07H 21/04

(52) U.S. Cl. 514/44 A; 435/375; 536/24.5

(57) ABSTRACT

The present invention provides methods for modulating translation of an mRNA using antisense oligonucleotides. The methods result in the stimulation or inhibition of a change in reading frame or stop codon readthrough during translation.

### CROSS-REFERENCE TO RELATED APPLICATION

This application claims the benefit of U. S. Provisional Patent application Ser. No. 60/599,089, filed Aug. 3, 2004, the entirety of which is incorporated by reference.

### STATEMENT REGARDING FEDERALLY SPONSORED RESEARCH OR DEVELOPMENT

Work described herein was supported in part by a grant from the National Institute of Health, grant number R01 NS43264. The United States Government may have certain rights in the invention.

### TECHNICAL FIELD

The invention relates to the field of biotechnology and antisense technology, more particularly to the use of antisense sequences to modulate translation.

### BACKGROUND

The standard rules of genetic read-out are well characterized. In its most basic sense, a protein is generated by reading (translating) the nucleotides of an mRNA strand in three nucleotide sets, or codons. Each codon specifies the addition of a particular amino acid to a growing polypeptide chain or an instruction to stop the process. The order of the nucleotides in the mRNA thus directly specifies the content and length of a polypeptide. Additionally, because codons are made up of three nucleotides, mRNA has three potential codon sets or reading frames. For example, translation of the sequence AAABBBCCCDDD may start at the first A with the resulting codons being AAA BBB CCC DDD, at the second A with the resulting codons being AAB BBC CCD DD, or at the third A with the resulting codons being ABB BCC CDD D. Each of these different utilizations of the sequence is referred to as a reading frame. In the majority of cases, only one reading frame is translated into a protein by the cell. This reading frame is known as the 0 reading frame. Events that switch translation to another reading frame are know as “frameshifting” events. For example, translation may utilize AAA as a codon, but due to a +1 frameshift event, the next codon will be read in the +1 reading frame as BBC. A −1 frameshift event would cause the next codon to be read in the −1 reading frame as ABB. However, because the content of polypeptides is extremely important for cellular function, unwanted frameshifting can have disastrous consequences.

One way that frameshifts occur is by mutations that delete or insert one or more nucleotides into a coding sequence. As long as the insertion or deletion is not a multiple of three, the reading frame is altered. Such mutations are known to result in numerous genetic diseases including forms of Duchenne and Becker muscular dystrophies, Ataxia telangiectasia, Cystic fibrosis, Hurler's syndrome, Hypercholesterolemia, Colorectal Adenomatous Polyposis, Insulin-dependent diabetes mellitus, Walker-warburg syndrome, Alstrom syndrome, Wilson disease, and Werner syndrome. Indeed, small mutations and deletions account for approximately 20.5% of the mutations in the Human Gene Mutation Database. Antonarakis et al., 2000. More than 87% of these mutations result in a change of reading frame. Id.

Another type of mutation that can greatly affect the outcome of translation is called a nonsense mutation. In this kind of mutation a single base is changed so that a codon that once specified an amino acid now instructs a stoppage in translation. As such, these kinds of mutations can result in the production of truncated proteins and reduce or eliminate the ability of the proteins to function. Nonsense mutations account for approximately 12% of all recorded mutations in the human genome and thus represent a major source of genetic disease in the human population. Id.

Therapeutic approaches to diseases caused by frameshift and nonsense mutations have been very limited and difficult to implement. As such, there is a need in the art for a method of treating such mutations/diseases.

### DISCLOSURE OF INVENTION

The present invention encompasses the use of antisense oligonucleotides to modulate protein translation. More particularly, it is demonstrated herein that antisense oligonucleotides have the capability to modulate ribosomal frameshifting and stop codon readthrough.

One aspect of the invention relates to a method of “correcting” of frameshift and nonsense mutations during translation. In essence, frameshift mutations may be corrected by inducing a compensating shift upstream, downstream, and/or at the site of the mutation. Nonsense mutations may be corrected by promoting a situation where an amino acid is inserted at a stop codon rather than terminating translation. Nonsense mutations may also be corrected by “shifting around” the stop codon, e.g., by shifting the reading frame so as to skip the stop codon.

Another aspect of the invention relates to the targeting of proteins involved in a disease process where the disease is caused by frame shift mutations and/or nonsense mutations. For example, the invention provides a method wherein a compensating frameshift event is induced in the translation of a gene having a frameshift mutation, thus restoring sufficient protein function to treat the disease.

A further aspect of the invention relates to the targeting of proteins involved in a disease process where the disease is not caused by a frame shift and/or a nonsense mutation. For example, the invention provides a method of down-regulating protein production, for example, by inducing a frameshift event in the translation of a protein not having a frameshift mutation, thus reducing protein levels and/or function to ameliorate a disease.

An additional aspect of the invention relates to the down-regulating the expression of a gene/gene product of interest. For example, the invention provides a method of down-regulating protein production, for example, by inducing a frameshift event in the translation of a protein not having a frameshift mutation, thus reducing protein levels and/or function for research purposes. The following embodiments are meant to be illustrative of the invention and are in no way intended to limit the invention to the embodiments disclosed herein.

According to one embodiment of the invention, an antisense oligonucleotide is used to restore proper translation of an mRNA produced from a mutated gene. The mutation an insertion or deletion of any size which is not a multiple of three which results in a shift in translation to an incorrect reading frame; either the −1 or +1 reading frame relative to the normal 0 reading frame. As will be recognized by one of skill in the art, proper translation of an mRNA containing a frameshift mutation can be restored by promoting the total frameshift to a multiple of 3 or by reducing it to zero. Thus, for example, a frameshift mutation which causes the translational machinery to read into the +1 reading frame can be functionally corrected by inducing a compensatory −1 shift in reading frame during translation. This may be accomplished by the translational machinery moving (3N+2) nucleotides towards the 3′ end of the mRNA, or (3N+1) nucleotides towards the 5′ end of the messages, where N=0 or any whole number. Likewise, correction of a frameshift mutation which causes the translational machinery to read into the −1 reading frame can be restored by a compensatory +1 shift in reading frame during translation. Proper translation of a mutated mRNA having an improper stop codon can be accomplished by using an antisense oligonucleotide to promote a situation where an amino acid is inserted at the stop codon rather than terminating translation and/or inducing frameshift prior to the target stop codon and a compensatory frameshift after the stop codon.

A further embodiment of the invention contemplates using antisense oligonucleotides to restore proper translation of an mRNA containing a nonsense mutation. In one aspect of this embodiment, the stop codon is mistranslated by promoting a situation where an amino acid is inserted at a stop codon rather than terminating translation.

In another embodiment of the invention, antisense oligonucleotides may be used to disrupt the proper translation of proteins. In one aspect of this embodiment, an antisense oligonucleotide anneals to an mRNA in order to promote a frameshift into either the +1 or −1 reading frame relative to the normal 0 reading frame and/or a termination of translation.

A further embodiment encompasses the use of antisense oligonucleotides to modulate translation in a subject having a disease caused by a frameshift and/or nonsense mutation thereby treating the disease. This embodiment, in part, contemplates a method wherein a sample is taken from a subject and analyzed such that at least one disease causing frameshift and/or nonsense mutation is identified. Antisense oligonucleotides capable of modulating translation are designed to correct the mutation and are provided to the subject.

Another embodiment of the invention contemplates treating a disease or infection in a subject. Proteins involved in the disease or infective processes are identified and an antisense oligonucleotide capable of causing a frameshift in the translation of a protein involved in the disease process is then designed and provided to the subject.

In a further embodiment, an antisense oligonucleotide may be provided to a subject with or without an adjuvant and/or a carrier by any method known to those of skill in the art, including, but not limited to, site-specific injection, systemic injection, intravenously, orally, and/or topically.

A yet further embodiment encompasses a medicament comprising antisense oligonucleotides able to modulate translation and, optionally, a pharmaceutically acceptable carrier and/or adjuvant.

Another embodiment of the invention contemplates the use of multiple antisense oligonucleotides. Examples of the uses of multiple antisense oligonucleotides include, but are not limited to, two separate −1 reading frame shifts to correct a −1 frameshift mutation, a −1 frameshift prior to a nonsense mutation followed by a +1 frameshift after the nonsense mutation, and multiple antisense oligonucleotides to correct and/or create multiple defects.

In another embodiment a method for generating an antisense oligonucleotide in the production of a pharmaceutical composition or medicament is provided. The method comprising locating at least one target site, selecting a target site, producing an antisense oligonucleotide able to modulate translation at the target site, preparing a medicament and/or a pharmaceutical composition comprising the antisense oligonucleotide.

The embodiments of the invention contemplate the use of one or more antisense oligonucleotides, which may be composed of, but are not limited to, morpholino, PNA, 2′-O-methyl, phosphorothioate, DNA oligonucleotides, and/or combinations there of in a single antisense oligonucleotide.

In the embodiments of the invention, it may be advantageous to induce frameshifting at a slippery site or a rare codon site.

The length of the antisense oligonucleotide in the embodiments of the invention may be between 2 and 100 nucleotides in length, between 10 and 50 nucleotides in length, and/or about 25 nucleotides in length, for example, 17, 18, 19, 20, 21, 22, 23, 24, 25, 26, 27, 28, 29, 30, 31, 31, 32, and/or 33 nucleotides in length. Furthermore, as will by appreciated by a person of skill in the art using the guidance of this specification, in all embodiments of the invention, the antisense oligonucleotide anneals to some location on the same molecule as the target site for modulation, with the exact location depending on the embodiment and the conditions to be affected. For example, inducing a frameshift may be effected by binding an antisense oligonucleotide downstream of the targeted frameshift site. In an exemplary embodiment, the 3′ end of the antisense oligonucleotide binds about 0-16 nucleotides downstream, about 1-10 nucleotides downstream, −1, 0, 1, 2, 3, 4, 5, 6, 7, 8, and/or 9 nucleotides downstream.

Additionally, it may be advantageous in any embodiment of the invention for the G/C:A/U ratio of the antisense oligonucleotide to be about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

Splicing and/or the secondary structure of an mRNA may or may not be affected using an antisense oligonucleotide according to the invention. Additionally, the annealing of the antisense nucleotide to a complementary mRNA may or may not interfere with proteins interacting with the mRNA.

Optionally, the antisense oligonucleotides may be modified in a manner to improve properties such as, but not limited to, improved ability to modulate translation, delivery, facilitate cellular uptake, direct intracellular localization, and/or modulate pharmacokinetics.

### BRIEF DESCRIPTION OF DRAWINGS

FIG. 1 shows the Morpholino induced frameshifting on the UUU UUA frameshift site. p2LucU6A was transcribed and translated in rabbit reticulocyte lysate in the absence or presence of increasing amounts of antisense MOAB morpholino. Control (Contr.) morpholino is a morpholino oligonucleotide corresponding to the sense sequence of MOAB. SDS PAGE (4-12% Bis-tris polyacrylamide gel) of 35 S methionine labeled protein products from transcription and translation reactions is shown. Concentrations are shown in μM and the location of the full length frameshift product and non-frameshift termination product are shown. Average percent frameshifting (% F.S.) and standard.

FIG. 2 illustrates a determination of optimal distance between an antisense morpholino oligonucleotide and −1 frameshift site using different antisense morpholino oligonucleotides. Spacer length effect on morpholino induced frameshifting. p2LucU6A was transcribed and translated in rabbit reticulocyte lysate in the absence or presence of increasing amounts of antisense morpholinos MOAD, MOAC, MOAB, MOAA, or MOA-1 which anneal 7, 5, 3, 1 or −1 nucleotides downstream of the frameshift site respectively. Control (Contr.) morpholino is a morpholino oligonucleotide corresponding to the sense sequence of MOAB. SDS PAGE (4-12% Bis-tris polyacrylamide gel) of 35 S methionine labeled protein products from transcription and translation reactions is shown. Each morpholino was included at a concentration of 1 μM. The location of the full length frameshift product and non-frameshift termination product are shown. Average percent frameshifting (% F.S.) and standard deviations (+/−) from the mean are shown below each lane of the gel.

FIG. 3 illustrates the effect of spacer length on morpholino induced frameshifting. Plasmids p2LucU6A-0, p2LucU6A, p2LucU6A-6, and p2LucU6A-9 were transcribed and translated in rabbit reticulocyte lysate in the absence or presence of increasing amounts of antisense morpholino MOAB which anneals 0, 3, 6, and 9 nucleotides downstream of the frameshift site for each construct respectively. SDS PAGE (4-12% Bis-trispolyacrylamide gel) of 35 S methionine labeled protein products from transcription and translation reactions is shown. MOAB was included at a concentration of 1 μM. The location of the full length frameshift product and non-frameshift termination product are shown. Average percent frameshifting (% F.S.) and standard deviations (+/−) from the mean are shown below each lane of the gel.

FIG. 4 illustrates the specificity of morpholino induced frameshifting. p2LucU6A was transcribed and translated in rabbit reticulocyte lysate in the absence or presence of antisense morpholinos, MOAB, MOA dmm3, MOA dmm4, or MOA dmm5 which contain 0, 3, 4, or 5 mismatches respectively. Each morpholino is added at a concentration of 1 μM. Control (Contr.) morpholino is a morpholino oligonucleotide corresponding to the sense sequence of MOAB. SDS PAGE (4-12% Bis-tris polyacrylamide gel) of 35 S methionine labeled protein products from transcription and translation reactions is shown. The location of the full length frameshift product and non-frameshift termination product are shown. Average percent frameshifting (% F.S.) and standard deviations (+/−) from the mean are shown below each lane of the gel.

FIG. 5 illustrates the effect of sequence composition on morpholino induced frameshifting. p2LucU6A, p2LucU6A A:T, and p2LucU6A G:C were transcribed and translated in rabbit reticulocyte lysate in the absence or presence of the complementary antisense morpholinos, MOAB, MOA A:T, and MOA G:C respectively. Control (Contr.) morpholino is a morpholino oligonucleotide corresponding to the sense sequence of MOAB. Each morpholino is added at a concentration of 1 μM. IF=p2LucU6A in which the 6 Us have been deleted. SDS PAGE (4-12% Bis-tris polyacrylamide gel) of 35 S methionine labeled protein products from transcription and translation reactions is shown. The location of the full length frameshift product and non-frameshift termination product are shown. Average percent frameshifting (% F.S.) and standard deviations (+/−) from the mean are shown below each lane of the gel.

FIG. 6 illustrates the effect of the heptanucleotide motif on morpholino induced frameshifting. p2LucAAAUUUA, p2LucGGGAAAC, p2LucUUUAAAC, p2LucAAAAAAC, p2LucAAAAAAG, and p2LucAAAAAAU were transcribed and translated in rabbit reticulocyte lysate in the presence of the complementary antisense morpholino, MOAB. Control (C) morpholino is a morpholino oligonucleotide corresponding to the sense sequence of MOAB. Each morpholino is added at a concentration of 1 μM. SDS PAGE (4-12% Bis-tris polyacrylamide gel) of 35 S methionine labeled protein products from transcription and translation reactions is shown. The location of the full length frameshift product and non-frameshift termination product are shown. Average percent frameshifting (% F.S.) and standard deviations (+/−) from the mean are shown below each lane of the gel.

FIG. 7 illustrates the effect of antisense oligonucleotide chemistry on frameshift induction. p2lucU6A was transcribed and translated in rabbit reticulocyte lysate in the presence of increasing amounts of complementary RNA, phosphorothioate, or 2′-O-Methyl antisense oligonucleotides. SDS PAGE (4-12% Bis-tris polyacrylamide gel) of 35 S methionine labeled protein products from transcription and translation reactions is shown. The concentration of oligonucleotide (uM) is shown above each gel lane, and average percent frameshifting (% F.S.) and standard deviations (+/−) from the mean are shown below each lane of the gel.

FIG. 8 illustrates the effect of antisense oligonucleotide chemistry on −1 frameshift induction in cells. p2luc-U6A-0 and MOAB were transfected into HEK 293 cells using Lipofectamine 2000. A histogram summarizing the results of the experiment is shown. Experimental cell cultures of HEK 293 cells were subjected to lipofectamine various amounts of MOAB as indicated on the x-axis. The percent frameshifting is shown along the y-axis.

FIG. 9 illustrates the effect of antisense oligonucleotide chemistry on +1 frameshift induction. p2luc+1 was transcribed and translated in rabbit reticulocyte lysate in the presence of 2 μm 2′-O-Methyl antisense oligonucleotides AZ1A, AZ1B, or AZ1C and/or 0.4 mM spermidine. A histogram summarizing the results of the experiment is shown. The percent frameshifting is shown along the y-axis. Noted along the x-axis are the different treatments that a particular example was subjected to.

### MODES FOR CARRYING OUT THE INVENTION

As used herein, a “frameshift,” “frame shifting,” “frameshift event” or other similar terms means a change in reading frame during the translation of an mRNA.

As used herein, a “frameshift mutation” means a mutation in an nucleic acid sequence that results in the normal reading frame being shifted into either the +1 or the −1 reading frame such that translation of an mRNA having a frameshift mutation results in a departure from the normal or wild-type reading frame.

As used herein, an “mRNA” means any nucleic acid molecule that can be translated into a protein.

As used herein, “treating” or “treatment” does not require a complete cure. It means that the symptoms of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease.

As used herein, target sites are located on an mRNA molecule where translation can be modulated or induced by the presence of an antisense oligonucleotide via frameshifting and/or stop codon readthrough. The mRNA molecule to which an antisense oligonucleotide anneals may be any mRNA molecule.

For the sake of brevity, “analysis of an mRNA of interest,” or other such references includes the analysis of genomic sequences, cDNA sequences or other sequences that directly or indirectly provide information regarding the mRNA sequence of interest.

One exemplary embodiment of a target site is a codon specifying a rare amino acid, charged tRNA, tRNA, and/or stop codon with an abundant codon in the +1 frame having an abundant amino acid, charged tRNA, and/or tRNA. As will be appreciated by one of skill in the art, a stop codon may be considered to be a rare codon it is typically slow to decode. This embodiment of a target site will hereinafter be referred to as a “rare codon site.” As will be apparent to one of skill in the art, there are species specific patterns to codon usage. As such, the abundant and/or rare codons, charged tRNAs, and tRNAs vary from organism to organism and are either known in the art or may be determined by a person of skill in the art using routine methods and techniques.

A further non-limiting embodiment of target site is a site comprising X XXY YYZ, wherein XXX is selected from the group consisting of GGG, AAA, UUU, and/or CCC; YYY is selected from the group consisting of AAA and/or UUU; and Z is selected from the group consisting of A, U, and/or C. This embodiment of a target site will hereinafter be referred to as a “slippery site.” In another embodiment, the target site is not AAAAAAA or UUUUUUU.

Further non-limiting embodiments of a target site include the stop codons UAA, UGA, and UAG. The antisense oligonucleotides may bind 3′, 5′, or directly to the target site.

Multiple considerations may be evaluated in determining a preferred target site. Such considerations may include, but are not limited to: distance from a mutation to be repaired, location up or down-stream from a mutation, the importance of information that is going to be decoded due to a frameshift at the target site, the presence of a stop codon in the material to be decoded, whether an antisense oligonucleotide optimized to promote frameshifting at a target site would anneal to one or more other mRNAs, and/or the ability of a specific target site to support frameshifting or stop codon readthrough.

### Antisense Oligonucleotides

The present invention provides for antisense oligonucleotides that base pair specifically with bases present on the mRNA molecule having a target site to be modulated. The antisense oligonucleotides will typically comprise purine and/or pyrimidine bases. Typically, the bases of the present invention are adenine, guanine, cytosine, thymidine, inosine, and/or uracil.

Base-pairing or binding between two or more bases may be accomplished by pair interactions including, but not limited to, Watson-Crick base-pairing, Hoogstein base-pairing, and/or reverse Hoogstein base-pairing. As a consequence of the precise nature of these types of base pairing interactions, antisense oligonucleotides can be designed to anneal to any predetermined sequence of a nucleic acid molecule.

The bases can be modified by, for example, the addition of substituents at, or modification of one or more position, for example, on the pyrimidines and purines. The addition of substituents may or may not saturate a double bond, for example, of the pyrimidines and purines. Examples of substituents include, but are not limited to, alkyl groups, nitro groups, halogens, and/or hydrogens. The alkyl groups can be of any length, preferably from one to six carbons. The alkyl groups may be saturated or unsaturated; and can be straight-chained, branched or cyclic. The halogens may be any of the halogens including, but not limited to, bromine, iodine, fluorine, and/or chlorine.

Further modification of the bases can be accomplished by the interchanging and/or substitution of the atoms in the bases. Non-limiting examples include: interchanging the positions of a nitrogen atom and a carbon atom in the bases, substituting a nitrogen and/or a silicon atom for a carbon atom, substituting an oxygen atom for a sulfur atom, and/or substituting a nitrogen atom for an oxygen atom. Other modifications of the bases include, but are not limited to, the fusing of an additional ring to the bases, such as an additional five or six membered ring. The fused ring may carry various further groups.

Specific examples of modified bases include, but are not limited to, 2,6-diaminopurine, 2-aminopurine, pseudoisocytosine, E-base, thiouracil, ribothymidine, dihydrouridine, pseudouridine, 4-thiouridine, 3-methylcytidine, 5-methylcytidine, N⁶-methyladenosine, N⁶-isopentenyladenosine, -methylguanosine, queuosine, wyosine, etheno-adenine, etheno-cytosine, 5-methylcytosine, bromothymine, azaadenine, azaguanine, 2′-fluoro-uridine, and 2′-fluoro-cytidine.

The bases are attached to a molecular backbone. Examples of molecular back bones include, but are not limited to, ribose, 2′-O-alkyl ribose, 2′-O-methyl ribose, 2′-O-allyl ribose, deoxyribose, 2-deoxyribose, morpholino, and/or peptide backbones. The backbone may comprise sugar and/or non-sugar units. These units may be joined together by any manner known in the art.

The units may be joined by linking groups. Some examples of linking groups include, but are not limited to, phosphate, thiophosphate, dithiophosphate, methylphosphate, amidate, phosphorothioate, methylphosphonate, phosphorodithioate, and/or phosphorodiamidate groups.

Alternatively, the units may be joined directly together. An example includes, but is not limited to, the amide bond of, for example a peptide backbone.

A sugar backbone may comprise any naturally occurring sugar. Examples of naturally occurring sugars include, but are not limited to, ribose, deoxyribose, and/or 2-deoxyribose.

A potential disadvantage of an antisense oligonucleotide having naturally-occurring sugar units as the back bone may be cleavage by nucleases. Cleavage of the antisense oligonucleotide might occur with the antisense oligonucleotide in a single-stranded form, and/or upon specifically binding to an mRNA molecule.

Accordingly, the sugar units of a backbone may be modified such that the modified sugar backbone is resistant to cleavage. The sugars of a backbone may be modified by methods known in the art, for example, to achieve resistance to nuclease cleavage. Examples of modified sugars include, but are not limited to, 2′-O-alkyl riboses, such as 2′-O-methyl ribose, and 2′-O-allyl ribose. The sugar units may be joined by phosphate linkers. Typical sugar units of the invention may be linked to each other by 3′-5′,3′-3′, or 5′-5′ linkages. Additionally, 2′-5′ linkages are also possible if the 2′ OH is not otherwise modified.

A non-sugar backbone may comprise any non-sugar molecule to which bases can be attached. Non-sugar backbones are known in the art. Examples include, but are not limited to, morpholino and peptide nucleic acids (PNAs). A morpholino backbone is made up of morpholino rings (tetrahydro-1,4-oxazine) and may be joined by non-ionic phosphorodiamidate groups. Modified morpholinos known in the art may be used in the present invention.

PNAs result when bases are attached to an amino acid backbone by molecular linkages. Examples of such linkages include, but are not limited to, methylene carbonyl, ethylene carbonyl, and ethyl linkages. The amino acids can be any amino acid, natural or non-natural, modified or unmodified and are preferably alpha amino acids. The amino acids can be identical or different from one another. One non-limiting example of a suitable amino acids include amino alkyl-amino acids, such as (2-aminoethyl)-amino acid.

Examples of PNAs include, but are not limited to, N-(2-aminoethyl)-glycine, cyclohexyl PNA, retro-inverso, phosphone, propinyl, and aminoproline-PNA. PNAs can be chemically synthesized by methods known in the art. Examples include, but are not limited to, modified Fmoc and/or tBoc peptide synthesis protocols.

In addition to the above mentioned uniform antisense oligonucleotides, it will now be apparent to one of skill in the art that multiple types of backbone can be mixed in a single antisense oligonucleotide. For example, a single antisense oligonucleotide may contain one or more 2′-O-methyl nucleotides, one or more morpholinos, one or more RNA nucleotides, and one or more PNAs.

The length of the antisense oligonucleotide is not critical, as long as the length is sufficient to hybridize specifically to the target site. For example, the base paring segment may have from about two to about one hundred bases, from about ten to fifty bases, about twenty five bases, or any individual number between about 16 and about 35.

Various factors may be considered when determining the length of the antisense oligonucleotide, such as target specificity, binding stability, cellular transport and/or in vivo delivery. Antisense oligonucleotides should be long enough to stably bind to the mRNA of interest. Also, the antisense oligonucleotides should be long enough to allow for reasonable binding specificity as a shorter sequence has a higher probability of occurring elsewhere in the genome than a longer sequence. Further considerations related to the length of an antisense oligonucleotide include, the efficiency of in vivo or ex vivo delivery, stability of the antisense oligonucleotide in vivo or in vitro, and/or the stability of the mRNA of interest bound or unbound by an antisense oligonucleotide.

The antisense oligonucleotides may be modified to optimize their use in various applications. Optimization may include, but is not limited to, one or more modification to improve delivery, cellular uptake, intracellular localization, and/or pharmacokinetics. One manner in which the antisense oligonucleotides may be modified is by the addition of specific signal sequences. Examples include, but are not limited to, nuclear retention signals, nuclear localization signals, and/or sequences that promote transport across cell membranes, the blood brain barrier, and/or the placental barrier. Specific examples include, but are not limited to, polylysine, poly(E-K), the SV40 T antigen nuclear localization signal, and/or the Dowdy Tat peptide. Sequences which localize antisense oligonucleotides to specific cell types are also contemplated.

Additionally, transport across cell membranes may be enhanced by combining the antisense oligonucleotides with one or more carriers, adjuvants, and/or diluents. Examples of such carriers, adjuvants, and/or diluents include, but are not limited to, water, saline, Ringer's solution, cholesterol and/or cholesterol derivatives, liposomes, lipofectin, lipofectamine, lipid anchored polyethylene glycol, block copolymer F108, and/or phosphatides, such as dioleooxyphosphatidylethanolamine, phosphatidyl choline, phosphatidylgylcerol, alpha-tocopherol, and/or cyclosporine. In many cases the antisense oligonucleotides may be mixed with one or more carriers, adjuvants, and/or diluents to form a dispersed pharmaceutical composition which may be used to treat a disease, such as a disease caused by a frameshift or nonsense mutation. See, e.g., Remington's Pharmaceutical Sciences; Goodman and Gilman's The Pharmacologic Basis of Therapeutics; Current Protocols in Molecular Biology. It would be apparent to a person of ordinary skill in the art that such a dispersed composition may also be used to disrupt the proper translation of genes involved in disease or infective processes.

Antisense oligonucleotides may also be linked to protein domains that enhance cellular uptake. Examples include, but are not limited to, the N-terminus of HIV-TAT protein and/or peptides derived from the Drosophila Antennapedia protein.

The antisense oligonucleotides, with or without an adjuvant and/or a carrier, may be administered to a subject in any manner that will allow the antisense oligonucleotides to modulate translation. Examples include, but are not limited to, site-specific injection, systemic injection, and/or administration intravenously, orally, and/or topically. Subjects contemplated by the invention include, but are not limited to, bacteria, cells, cell culture systems, plants, fungi, animals, nematodes, insects, and/or mammals, such as humans.

Antisense oligonucleotides may be further optimized to provide the greatest amount of translational modulation. Typically, such optimization will result in an antisense oligonucleotide that has a 3′ end which anneals 0-16, 1-5, -7, -6, -5, -4, -3, -2, -1, 0, 1, 2, 3, 4, 5, 6, 7, 8, 9, 10, 11, 12, 13, 14, 15, and/or 16 nucleotides 3′ of the target site and has very few or no mismatched bases when annealed to an mRNA. Antisense oligonucleotides may also be optimized as to their G/C content and for the G/C:A/T ratio.

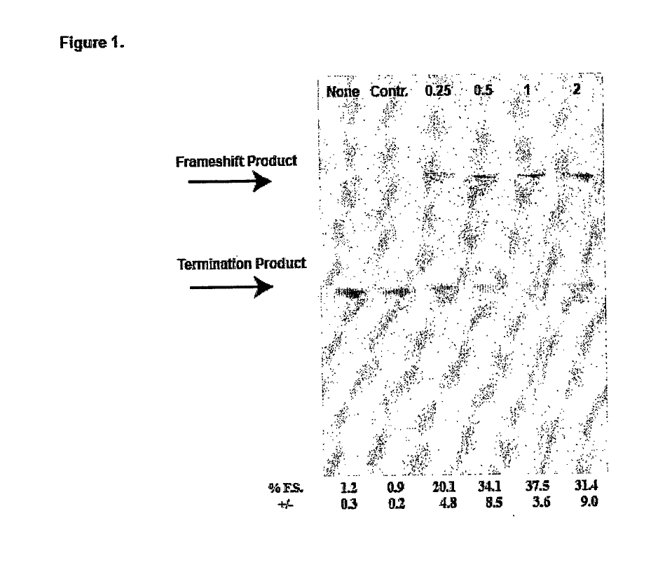

Antisense oligonucleotides according to the invention may increase frame shifting by about 5%, about 10%, about 15%, about 20%, about 25%, about 30%, about 40% and/or about 45%. Using the antisense oligonucleotides according to the invention may also increase frame shifting by at least about 5%, about 10%, about 15%, about 20%, about 25%, about 30%, about 45% and/or about 50%, for example, as illustrated in FIGS. 1, 2, 7, and Examples 1 and 2.

### Modulation of Translation

As referred to in this application, modulation of translation involves either a change in reading frame during translation or stop codon readthrough. The change in reading frame can take place on normal or mutated mRNAs and encompasses shifts to both the −1 or +1 reading frame relative to the normal 0 reading frame. As would be apparent to one of skill in the art, a shift in reading frame may encompass (3N+1) or (3N+2) nucleotides being not translated or translated twice, where N=any whole number. For example, a shift to the +1 reading frame may involve a ribosome moving backward two nucleotides or a shift to the −1 reading frame may involve a ribosome skipping forward 50 nucleotides. Ivanov et al., 1998; Weiss et al., 1990. Stop codon readthrough involves increasing the likelihood of continuing translation in a reading frame past a codon that normally terminates translation.

In an embodiment of the invention, an antisense oligonucleotide is used to compensate for a mutation resulting part of the mRNA of interest being translated in the −1 reading frame relative to the normal reading frame, such as a (3N+1) base insertion or a (3N+2) deletion in the coding sequence of an mRNA of interest, where N=0 or any whole number. The mRNA of interest is analyzed for the presence of target sites. A preferred target site, for example a rare codon site is then selected. An optimized antisense oligonucleotide is then designed and provided to a subject so as to stimulate a +1 frameshift at the preferred target site.

In a further embodiment of the invention, an antisense oligonucleotide is used to compensate for a mutation resulting part of the mRNA of interest being translated in the +1 reading frame relative to the normal reading frame, such as a (3N+1) base deletion or a (3N+2) insertion into the coding sequence of an mRNA of interest, where N=0 or any whole number. Optionally, the mRNA of interest may be analyzed to identify the presence of target sites. A preferred target site, for example a slippery site is then selected. An optimized antisense oligonucleotide is then designed and provided to a subject so as to stimulate a −1 frameshift at the preferred target site.

In another embodiment, an antisense oligonucleotide is used to correct a nonsense mutation in the normal reading frame of an mRNA of interest. Optionally, the mRNA of interest may be analyzed to identify the location of the nonsense mutation. An optimized antisense oligonucleotide is then designed and provided to a subject so as to stimulate the insertion of an amino acid at the stop codon thus resulting in readthrough of the stop codon at the location of the nonsense mutation.

In a further embodiment, a combination of +1 and −1 shifts in reading frame are used to “shift around” a nonsense mutation in the normal reading frame of an mRNA of interest. Optionally, the mRNA of interest may be analyzed to identify the location of target sites, for example, rare codon and slippery sites. A first target site is then selected upstream or downstream of the nonsense mutation, and a second target site is selected such that the location of the nonsense mutation is between the first and second target sites and that the second target site is capable of stimulating a shift in reading frame in the opposite manner of the first target site. For example, a first target site may be a rare codon site downstream from the nonsense mutation. In such a case, the second target site would be upstream of the nonsense mutation and be capable of stimulating a −1 shift in reading frame, for example, a slippery site. Optimized antisense oligonucleotides are then designed for each target site and provided to a subject so as to stimulate frameshifting at the target sites.

In an additional embodiment, an antisense oligonucleotide is used to disrupt the normal reading frame during the translation of an mRNA of interest. Optionally, the mRNA of interest may be analyzed to identify the location target sites. A preferred target site, for example a slippery site and/or a rare codon site, is then selected. An optimized antisense oligonucleotide is then designed and provided to stimulate a frameshift at the target site. Such disruption of a the normal reading frame may be useful for, for example, but no limited to, disrupting genes involved in a disease or infectious processes. Examples of such genes include, but are not limited to, oncogenes, inflammatory genes, signaling molecules, secondary messengers, cytokines, hormones, receptors, viral genes, bacterial genes, and/or prions. Such disruption may by be further useful for, for example, to study the effects of reduced protein expression.

In a further embodiment, an antisense oligonucleotide is used to treat a subject having a viral infection. A virus infecting a subject is identified and the viral genome or mRNA is analyzed to identify the location of target sites. A preferred target site, for example a −1 and/or a rare codon site, is then selected. An optimized antisense oligonucleotide is then designed and provided to the subject to stimulate a frameshift at the target site. It will be appreciated by one of skill in the art that once an optimized antisense oligonucleotide is designed for a specific virus, repeated analysis and design are no longer required. It will be further appreciated that cocktails of multiple antisense oligonucleotides may be provided to target a broad spectrum of viruses and strains of viruses without a specific identification or diagnosis.

In an additional embodiment, an antisense oligonucleotide is used to affect a virus by disrupting normal frameshifting. This may be accomplished, for example, by disrupting a pseudoknot or other secondary structure that normally affects frame shifting, for example, the pseudoknot between the gag and pol genes of the HIV virus. Optionally, the mRNA of a virus may be examined to identify putative secondary structure downstream from a target site. An antisense oligonucleotide which anneals to the sequences forming the secondary structure is provided to a subject in which the virus may be present. This embodiment may be useful, for example, for treating a subject infected with a virus or for identifying secondary structure that contributes to normal frameshifting.

In an additional embodiment, an antisense oligonucleotide comprises part of a medicament designed to modulate translation and, optionally, one or more pharmaceutically acceptable carrier and/or adjuvant. Examples of such carriers, diluents and/or adjuvants, include, but are not limited to, water, saline, Ringer's solution, cholesterol and/or cholesterol derivatives, liposomes, lipofectin, lipofectamine, lipid anchored polyethylene glycol, block copolymer F108, and/or phosphatides, such as dioleooxyphosphatidylethanolamine, phosphatidyl choline, phosphatidylgylcerol, alpha-tocopherol, and/or cyclosporine. For additional carriers and adjuvants, as well as methods of producing a medicament, see, e.g., Goodman and Gilman's The Pharmacologic Basis of Therapeutics; Remington's Pharmaceutical Sciences; Mann et al., 2001.

In all embodiments of the invention, it will be appreciated by one of skill in the art that a −1 frameshift event has an equivalent effect on the current reading frame as two +1 frameshift events and that a +1 frameshift event has an equivalent effect on the current reading frame as two −1 frameshift events. Furthermore, it will be appreciated by one of skill in the art that in all embodiments of the invention multiple target sites may be targeted simultaneously by multiple antisense oligonucleotides so as to insure a maximal amount of translational modulation.

In all embodiments of the invention, the antisense oligonucleotide may be provided to any subject in which a modulation of translation is required. Such subjects include, but are not limited to, in vitro culture systems, bacteria, plants, fungi, animals, nematodes, insects, amphibians, and/or mammals such as humans. As would be apparent to one of skill in the art, antisense oligonucleotides have proven efficacy in a number of biological systems ranging from, for example but not limited to, cell free rabbit reticulocyte systems (Taylor et al., 1996), cellular culture systems (Dunckley et al., 1998), and/or live animal systems (Mann et al., 2001). Additionally, in all embodiments of the invention, an antisense oligonucleotide may be provided to a subject with or without an adjuvant and/or carrier by any method known to those of skill in the art, including, but not limited to, site-specific injection, systematic injection, intravenously, orally, and/or topically.

The invention is further described by way of the following illustrative examples.

### EXAMPLES

### Example 1−1 Frameshifting

The ability of morpholino antisense oligonucleotides to induce ribosomal frameshifting was determined by in vitro transcription and translation of the dual luciferase reporter vector, p2Luc, in the presence or absence of morpholino oligonucleotides. P2Luc contains the renilla and firefly luciferase genes on either side of a multiple cloning site, and can be transcribed using the T7 promoter located upstream of the renilla luciferase gene (Grentzmann et al., 1998). Sequences containing a target site were cloned between the two reporter genes such that translation of the downstream firefly luciferase gene and the production of full length protein requires a −1 shift in reading frame during translation. The following p2Luc constructs were created: the 0 reading frame of the target site is shown

| | |
|---|---|
| p2Luc-U6A -0 | |
| TCG ACG AAT TTT TTA TGG | (SEQ ID NO: 1) |
| p2Luc-U6A | |
| TCG ACG AAT TTT TTA GGG TGG | (SEQ ID NO: 2) |
| p2Luc-U6A -6 | |
| TCG ACG AAT TTT TTA GGG CAG TGG | (SEQ ID NO: 3) |
| p2Luc-U6A -9 | |
| TCG ACG AAT TTT TTA GGG CAG AGC TGG | (SEQ ID NO: 4) |
| p2Luc-U6A A:T | |
| TCG AAT TTT TTA GGG ATA TAA | (SEQ ID NO: 5) |
| p2Luc-U6A G: | |
| CTCG AAT TTT TTA GGG GCG GGC | (SEQ ID NO: 6) |
| p2Luc-A6C | |
| TCG TCA AAA AAC TTG TGG | (SEQ ID NO: 7) |
| p2Luc-A6G | |
| TCG TCA AAA AAG TTG TGG | (SEQ ID NO: 8) |
| p2Luc-A6U | |
| TCG TCA AAA AAT TTG TGG | (SEQ ID NO: 9) |
| p2Luc-UUUAAAC | |
| TCG CCT TTA AAC CAG TGG | (SEQ ID NO: 10) |
| p2Luc-GGGAAAC | |
| TCG CAG GGA AAC GGA TGG | (SEQ ID NO: 11) |
| p2Luc-AAAUUUA | |
| TCG ACA AAT TTA TAG TGG | (SEQ ID NO: 12) |

The constructs were transcribed and translated in vitro with complementary morpholinos, using Rabbit Reticulocyte lysates in the presence of ³⁵S Methionine and analyzed by electrophoresis on SDS polyacrylamide gels. More specifically, the dual luciferase constructs described above were in some cases added directly to TNT Coupled Reticulocyte Lysate reactions as described (Promega). In other cases, the dual luciferase constructs were linearized with Pml-1 restriction enzyme prior to the production of capped mRNA by in vitro transcription reactions utilizing the mMessage mMachine Kit obtained from Ambion, Inc. In the latter case, 0.2 ug of capped mRNA was added to 6.6 μl of Rabbit Reticulocyte Lysate, 70 mM KCL, 0.02 mM of each amino acid except Methionine, 4 μCi of 35S methionine (1000 Ci/mmol) in a total of 10 ul. Proteins were separated by SDS polyacrylamide gel electrophoresis and the gels were fixed with 7.5% acetic acid and methanol for 20 minutes. After drying under vacuum, the gels were visualized using a Storm 860 phosphorimager (Molecular Dynamics) and radioactive bands quantified using ImageQuant software.

TABLE 1
Morpholino Oligonucleotides:

| | |
|---|---|
| Control MOA | ATCCTTCAACTTCCCTGAGCTCGAA |
| | (SEQ ID NO: 13) |
| MOA-1 | CAGGGAAGTTGAAGGATCCCACCCT |
| | (SEQ ID NO: 14) |
| MOAA | CTCAGGGAAGTTGAAGGATCCCACC |
| | (SEQ ID NO: 15) |
| MOAB | AGCTCAGGGAAGTTGAAGGATCCCA |
| | (SEQ ID NO: 16) |
| MOAC | CGAGCTCAGGGAAGTTGAAGGATCC |
| | (SEQ ID NO: 17) |
| MOAD | TTCGAGCTCAGGGAAGTTGAAGGAT |
| | (SEQ ID NO: 18) |
| MOAE | TCTTCGAGCTCAGGGAAGTTGAAGG |
| | (SEQ ID NO: 19) |
| MOA dmm3 | ACCTCAGCGAAGTTGAAGCATCCCA |
| | (SEQ ID NO: 20) |
| MOA dmm4 | ACCTCAGCGAAGTTGAAGCATCGCA |
| | (SEQ ID NO: 21) |
| MOA dmm5 | ACCTCAGCGAACTTGAAGCATCGCA |
| | (SEQ ID NO: 22) |
| MOA A:T | TCAGGGAAGTTGAAGGATCTTATAT |
| | (SEQ ID NO: 23) |
| MOA G:C | TCAGGGAAGTTGAAGGATCGCCCGC |
| | (SEQ ID NO: 24) |

Morpholino oligonucleotides (Table 1) were synthesized by Gene Tools, LLC. Oligonucleotides were re-suspended in H₂O and added directly to in vitro translations at the indicated concentrations. Mismatch nucleotides are shown in bold for dmm3, 4, and 5.

Percent frameshifting was calculated as the percent of full length (frameshift) product relative to the termination product and the full length product combined. The value of each product was corrected for the number of methionine codons present in the coding sequence. The reported values are the average of three independent points from a representative experiment.

### Increased Frameshifting

The ability of a morpholino antisense oligonucleotide to induce translational frameshifting at the highly shifty U UUU UUA sequence was initially examined. A single morpholino oligonucleotide, MOAB, was designed to anneal 3 nucleotides downstream of the shift site within the vector p2LucU₆A. Titration of the morpholino oligonucleotide into coupled transcription/translation reactions revealed a maximal frameshifting level of approximately 40% with 1 μM morpholino oligonucleotide—a 20 fold increase in frameshifting over that observed either in the absence of morpholino oligonucleotide or with a control sense morpholino oligonucleotide (FIG. 1). To verify that the morpholino oligonucleotide was activating ribosomal frameshifting and not transcription slippage, RNA was transcribed in the absence of morpholino oligonucleotide and added to reticulocyte lysate translations in the presence of 1 μM MOAB morpholino oligonucleotide. Frameshifting levels were increased by 20 fold as observed in coupled reactions demonstrating that the morpholino oligonucleotide acts to induce frameshifting during translation (data not shown).

### Spacer Effects

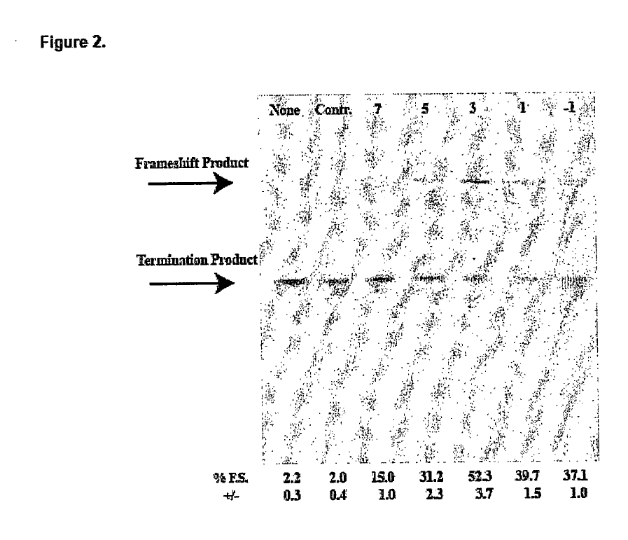

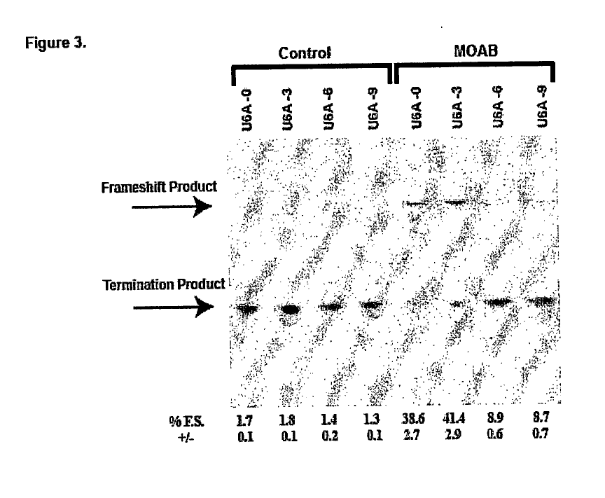

The spacer length between frameshift sites and downstream stimulators in programmed −1 frameshifting is important for optimal frameshift stimulation (Brierley et al., 1989; Kollmus et al., 1994). To determine the optimal spacer length between the shift site and the morpholino antisense oligonucleotide:RNA hybrid, morpholino antisense oligos MOA-1, MOAA, MOAB, MOAC, and MOAD (see Table 1) were designed to hybridize downstream from the U UUU UUA site in p2LucU₆A such that either −1, 3, 5, 7, or 9 nucleotides separate the last A of the shift site from the 3′ end of the morpholino oligonucleotide (“−1” hybridizes with the A of the U UUU UUA sequence). The greatest level of frameshifting was observed with a 3 nucleotide spacer (FIG. 2) but also occurred with the −1, 5, 7, and 9 nucleotide spacers. However, using this approach, the morpholino oligonucleotide sequence is by necessity altered at each location. In order to address the spacer effect without altering the morpholino sequence, three additional p2Luc constructs were produced p2LucU₆A-0, p2LucU₆A-6, p2LucU₆A-9 such that either 0, 6, or 9 nucleotides separate the last A of the U UUU UUA shift site from the first complementary base (3′ end) of the MOAB morpholino oligonucleotide. Spacer distances of 0 or 3 nucleotides stimulated frameshifting to approximately 40% whereas 6 and 9 nucleotide spacers stimulated frameshifting levels to just below 10% (FIG. 3). Therefore, the optimal spacer length was found to be between 0 and 5 nucleotides downstream of the shift site.

### Morpholino Oligonucleotide Specificity

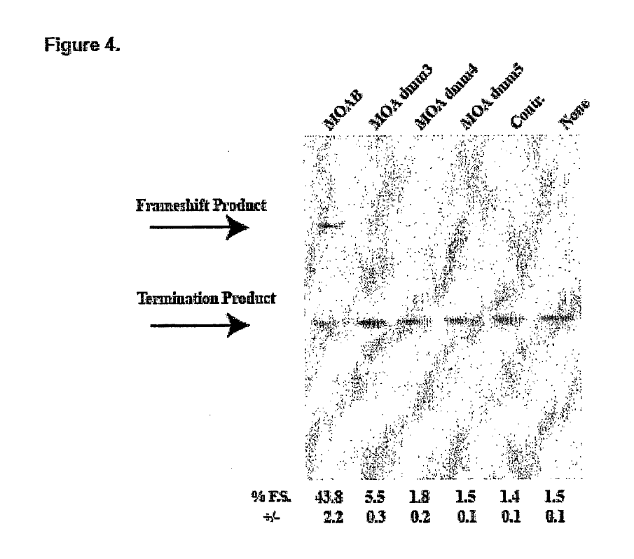

To test the annealing site sequence specificity for the action of morpholino oligonucleotides to induce frameshifting, we tested the effect of three morpholino oligonucleotides (MOA dmm3, MOA dmm4, and MOA dmm5 (see Table 1)) corresponding to the MOAB sequence but containing 3, 4, or 5 mismatched nucleotides respectively (See Materials and Methods). Three mismatches reduced frameshifting levels on the U UUU UUA shift site in in vitro transcriptions/translation reactions from approximately 40% to 5%, and additional mismatches reduced frameshifting levels to background levels (FIG. 4). These results demonstrate the sequence specificity of the antisense oligonucleotides relative to the annealing site and suggest that antisense oligonucleotides are unlikely to affect ribosome frame maintenance at non-target sites.

### Sequence Effects

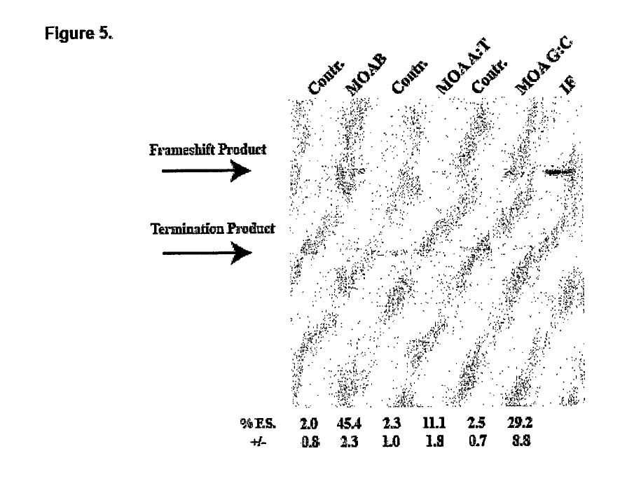

The sequence composition of the morpholino oligonucleotide:RNA hybrid may influence ribosome frameshifting due to increases in the thermodynamic stability of G:C rich sequences relative to those which are A:U rich. Two additional p2Luc constructs were made with the first 6 nucleotides after the 3 nucleotide spacer changed to be entirely A and U, or G and C nucleotides. Corresponding complementary morpholino oligonucleotides, MOA A:T and MOA G:C (see Table 1), were synthesized and tested in in vitro transcription and translation reactions for their ability to induce frameshifting on the U UUU UUA frameshift site. Interestingly, MOAB with 50% G:C composition gave higher frameshifting efficiencies (˜45%), than MOA A:T (˜10%) or MOA G:C (˜30%) (FIG. 5). These results demonstrate an antisense oligonucleotide:RNA hybrid sequence effect on ribosomal frameshifting and suggest that an intermediate thermodynamic stability may result in maximal frameshift induction.

### Heptanucleotide Shift Sites

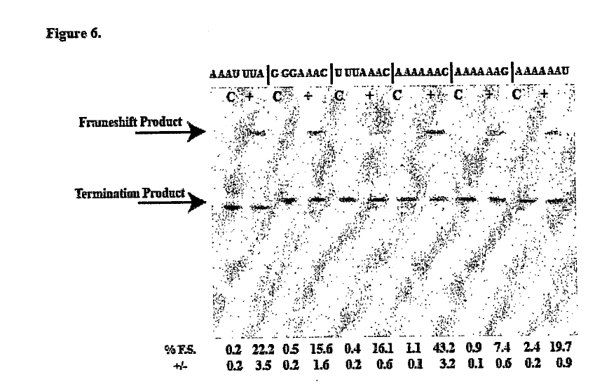

Morpholino oligonucleotide induced frameshifting was examined at six additional heptanucleotide frameshift motifs, A AAU UUA; G GGA AAC; U UUA AAC; A AAA AAC; A AAA AAG; and A AAA AAU. The complementary MOAB morpholino oligonucleotide was added to 1 μM in in vitro transcriptions/translations of p2LucAAAUUUA; p2LucGGGAAAC, p2LucUUUAAC, p2LucA₆C, p2LucA₆G, and p2LucA₆U (FIG. 6). Frameshifting on the A AAA AAC shift site was equivalent, ˜40%, to that observed at the U UUU UUA sequence, whereas changing the C to either a U or a G reduced frameshift levels to 20 and 7% respectively. Frameshifting at the A AAU UUA, G GGA AAC, and U UUA AAC sites ranged between 20 and 15%. Thus, frameshift stimulation is not unique to the U UUU UUA site and varies in efficiency depending upon the P- and A-site codons.

### Example 2. −1 Frameshifting with Multiple Types of Antisense Oligonucleotide

The ability of multiple kinds of antisense oligonucleotides to induce ribosomal frameshifting was determined by in vitro transcription and translation of the dual luciferase reporter vector, p2Luc-U6A, in the presence or absence of antisense oligonucleotides. P2Luc-U6A, is as described in Example 1. The production of full length protein incorporating both luciferase proteins requires a −1 shift in reading frame.

The construct was transcribed and translated in vitro with various amounts of complementary antisense oligonucleotides, using Rabbit Reticulocyte lysates in the presence of ³⁵S Methionine and analyzed by electrophoresis on SDS polyacrylamide gels. More specifically, the dual luciferase constructs described above were in some cases added directly to TNT Coupled Reticulocyte Lysate reactions as described (Promega). In other cases, the dual luciferase constructs were linearized with Pml-1 restriction enzyme prior to the production of capped mRNA by in vitro transcription reactions utilizing the mMessage mMachine Kit obtained from Ambion, Inc. In the latter case, 0.2 μg of capped mRNA was added to 6.6 ul of Rabbit Reticulocyte Lysate, 70 mM KCL, 0.02 mM each amino acid except Methionine, 4 μCi of 35S methionine (1000 Ci/mmol) in a total of 10 ul. Proteins are separated by SDS polyacrylamide gel electrophoresis and the gels are fixed with 7.5% acetic acid and methanol for 20 minutes. After drying under vacuum, the gels are visualized using a Storm 860 phosphorimager (Molecular Dynamics) and radioactive bands quantified using ImageQuant software.

Percent frameshifting was calculated as the percent of full length (frameshift) product relative to the termination product and the full length product combined. The value of each product is corrected for the number of methionine codons present in the coding sequence.

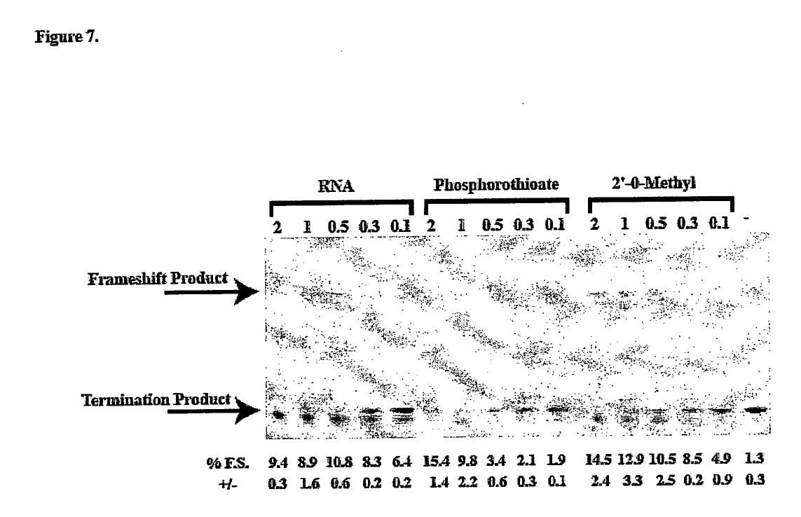

The ability of various types of antisense oligonucleotides to induce translational frameshifting at the highly shifty U UUU UUA sequence was examined. Antisense oligonucleotides were designed to anneal 3 nucleotides downstream of the shift site within the vector p2LucU₆A. Titration of the antisense oligonucleotides into coupled transcription/translation reactions revealed a maximal frameshifting level of approximately 40% with 1 μM antisense morpholino oligonucleotide—a 22 fold increase in frameshifting over that observed in the absence of antisense oligonucleotide (FIG. 7). Furthermore, antisense RNA, phosphorothioate, and 2′-O-methyl oligonucleotides all demonstrated a significant ability to stimulate −1 frameshifting when compared to control. Given the general usefulness of antisense oligonucleotides, one of skill in the art would appreciate that antisense oligonucleotides of this example would be effective in not only rabbit reticulocyte systems, but in cell culture and whole animal systems as well.

### Example 3. −1 Frameshifting in Cell Cultures

The ability of antisense oligonucleotides to induce ribosomal frameshifting is determined by in vitro transcription and translation of the dual luciferase reporter vector, p2Luc, in the presence or absence of antisense oligonucleotides. P2Luc is as described in Example 1. Sequences containing a target site are cloned between the two reporter genes such that translation of the downstream firefly luciferase reporter gene and the production of full length protein requires a −1 shift in reading frame during translation. The following p2Luc construct is created: 0 reading frame is shown:

| (SEQ ID NO: 25) | |
|---|---|
| p2LucU6A-0 | TCG ACG AAT TTT TTA TGG GAT C. |

The human embryonic kidney cell line, HEK 293, was obtained from ATCC and maintained as previously described (Howard et al., 2000) in the absence of antibiotics. Cells used in these studies were subcultured at 70% confluence and used between passages 7 and 15. Cells were transfected with the p2lucU6A reporter vector and MOAB antisense oligonucleotide using Lipofectamine 2000 reagent (Invitrogen) in a one-day protocol in which suspension cells are added directly to the DNA- and morpholino-lipofectamine complexes in 96-well plates. Cells were trypsinized, washed and added at a concentration of 4×10⁴ cells/well in 50 μl DMEM, 10% FBS. Transfected cells were incubated overnight at 37° in 5% CO₂, then 75 μl DMEM, 10% FBS were added to each well, and the plates were incubated an additional 48 hours.

Luciferase activities were determined using the Dual Luciferase Reporter Assay System (Promega). Relative light units were measured on an autoinjection luminometer (Turner Biosystems). Transfected cells were lysed in 12.5 μl lysis buffer and light emission was measured following injection of 25 μl of luminescence reagent. Frameshifting was calculated by comparing firefly:renilla luciferase ratios of experimental constructs with those of control constructs: (firefly experimental RLUs/renilla experimental RLUs)/(firefly control RLUs/renilla control RLUs)×100.

### Increased Frameshifting

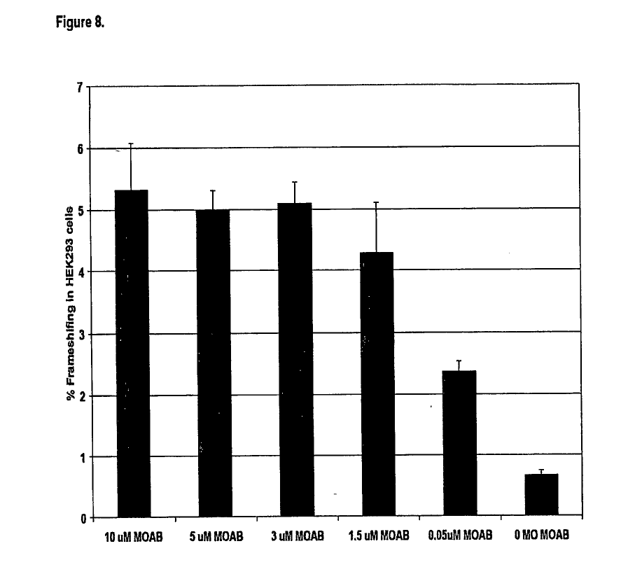

Addition of morpholino antisense oligonucleotide resulted in approximately 5% of the ribosomes to shift into the −1 reading frame (FIG. 8). This result demonstrates the antisense oligonucleotides can be used to suppress frameshift mutation in which a −1 frameshift during translation would restore the ribosome to the correct reading frame at or nearby the frameshift mutation.

### Example 4. +1 Frameshifting

The ability of antisense oligonucleotides to induce ribosomal frameshiffing is determined by in vitro transcription and translation of the dual luciferase reporter vector, p2Luc, in the presence or absence of antisense oligonucleotides. P2Luc is as described in Example 1. Sequences containing a target site are cloned between the two reporter genes such that translation of the downstream firefly luciferase reporter gene and the production of full length protein requires a +1 shift in reading frame during translation. The following p2Luc construct is created: 0 reading frame is shown:

| (SEQ ID NO: 26) | |
|---|---|
| p2Luc + 1 | TCG ACG TGC TCC TGA TGC CCC TGG ATC. |

The construct is transcribed and translated in vitro with complementary antisense oligonucleotides, using Rabbit Reticulocyte lysates in the presence of ³⁵S Methionine and analyzed by electrophoresis on SDS polyacrylamide gels. More specifically, the dual luciferase constructs described above are in some cases added directly to TNT Coupled Reticulocyte Lysate reactions as described (Promega). In other cases, the dual luciferase constructs are linearized with Pml-1 restriction enzyme prior to the production of capped mRNA by in vitro transcription reactions utilizing the mMessage mMachine Kit obtained from Ambion, Inc. In the latter case, 0.2 ug of capped mRNA is added to 6.6 ul of Rabbit Reticulocyte Lysate, 70 mM KCL, 0.02 mM each amino acid except Methionine, and 4 uCi of ³⁵S methionine (1000 Ci/mmol) in a total of 10 ul. Proteins are separated by SDS polyacrylamide gel electrophoresis and the gels are fixed with 7.5% acetic acid and methanol for 20 minutes. After drying under vacuum, the gels are visualized using a Storm 860 phosphorimager (Molecular Dynamics) and radioactive bands quantified using ImageQuant software.

TABLE 2
2′-O-Methyl Anitsense Oligonucleotides0

| | | |
|---|---|---|
| AZ1A | AGUUGAAGGAUCCAGGGGCA | (SEQ ID NO: 27) |
| AZ1B | GGAAGUUGAAGGAUCCAGGG | (SEQ ID NO: 28) |
| AZ1C | CAGGGAAGUUGAAGGAUCCA | (SEQ ID NO: 29) |

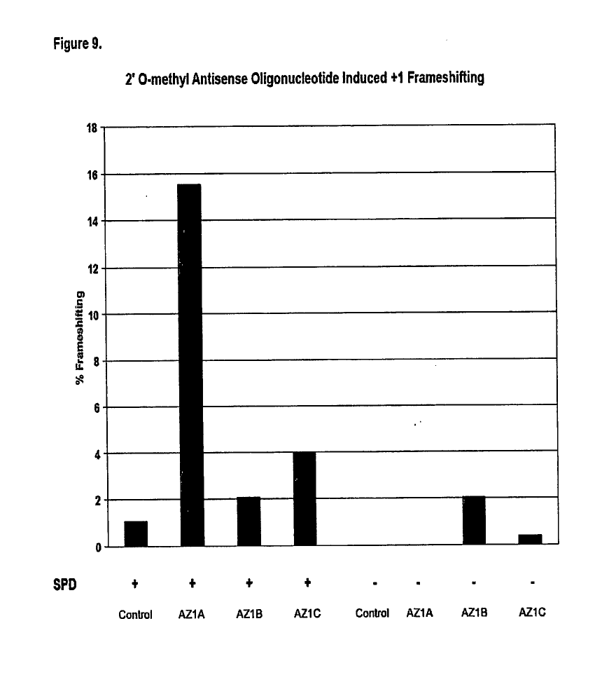

2′-O-methyl modified antisense oligonucleotides were synthesized by Integrated DNA Technologies (Coralville, Iowa). Antisense oligonucleotides AZ1A, AZ1B, and AZ1C (Table 2) were designed such that the 3′ end of the oligonucleotide was complementary to the first, fourth, and seventh nucleotide following the shift site respectively. The antisense oligonucleotides were added directly to the in vitro translations as indicated in FIG. 9 at a concentration of 2 μM, with or without 0.4 mM Spermidine. Samples were incubated for 1 hour at 32 degrees centigrade prior to separation by SDS polyacrylamide gel electrophoresis.

Percent frameshifting is calculated as the percent of full length (frameshift) product relative to the termination product and the full length product combined. The value of each product is corrected for the number of methionine codons present in the coding sequence and +1 frameshifting is identified.

### Increased Frameshifting

The highest level of frameshift induction was observed with the AZ1A antisense oligonucleotide which induced approximately 15% of the ribosomes to shift into the +1 reading frame in the presence of exogenously added spermidine (FIG. 9). In the absence of spermidine, AZ1B induced approximately 2% of the ribosomes to shift into the +1 reading frame. This result demonstrates that antisense oligonucleotides can be used to suppress a frameshift mutation in which a +1 frameshift during translation would restore the ribosome to the correct reading frame at or nearby the frameshift mutation.

### Example 5. +1 Frameshifting Due to a Rare Codon

The ability of antisense oligonucleotides to induce ribosomal frameshifting is determined by in vitro transcription and translation of the dual luciferase reporter vector, p2Luc, in the presence or absence of antisense oligonucleotides. P2Luc is as described in Example 1. Sequences containing a target site are cloned between the two reporter genes such that translation of the downstream firefly luciferase gene and the production of full length protein requires a +1 shift in reading frame during translation. The following p2Luc construct is created: 0 reading frame is shown:

| | |
|---|---|
| p2Luc-Rare | TCG CGA GAA TGG. (SEQ ID NO: 30) |

The second codon in the target site (CGA in this example) represents a relatively rare codon while the codon in the +1 reading frame (GAG in this example) specifies a relatively abundant codon. What is a rare or abundant codon will vary from species to species. Rare and/or abundant codons may be selected based on tRNA abundance or relative codon usage. See, e.g., Sharp et al., 1988; Gilis et al., 2001. The CGA codon specifying arginine is a relatively rare codon as the particular codon accounts for only 0.56% of codons in human protein. Alf-Steinberger, C., 1986. The codon in the +1 reading frame, GAG, specifies glutamic acid which is relatively abundant at 4.2% of codons.

The construct is transcribed and translated in vitro with complementary antisense oligonucleotides, using Rabbit Reticulocyte lysates in the presence of ³⁵S Methionine and analyzed by electrophoresis on SDS polyacrylamide gels. More specifically, the dual luciferase constructs described above are in some cases added directly to TNT Coupled Reticulocyte Lysate reactions as described (Promega). In other cases, the dual luciferase constructs are linearized with Pml-1 restriction enzyme prior to production of capped mRNA by in vitro transcription reactions utilizing the mMessage mMachine Kit obtained from Ambion, Inc. In the latter case, 0.2 ug of capped mRNA is added to 6.6 ul of Rabbit Reticulocyte Lysate, 70 mM KCL, 0.02 mM each amino acid except Methionine, and 4 uCi of ³⁵S methionine (1000 Ci/mmol) in a total of 10 ul. Proteins are separated by SDS polyacrylamide gel electrophoresis and the gels are fixed with 7.5% acetic acid and methanol for 20 minutes. After drying under vacuum, the gels are visualized using a Storm 860 phosphorimager (Molecular Dynamics) and radioactive bands quantified using ImageQuant software.

Percent frameshifting is calculated as the percent of full length (frameshift) product relative to the termination product and the full length product combined. The value of each product is corrected for the number of methionine codons present in the coding sequence and +1 frameshifting is identified.

### Example 6. Stop Codon Readthrough

The ability of antisense oligonucleotides to induce stop codon readthrough is determined by in vitro transcription and translation of the dual luciferase reporter vector, p2Luc, in the presence or absence of antisense oligonucleotides. P2Luc contains the renilla and firefly luciferase genes on either side of a stop codon, and can be transcribed using the T7 promoter located upstream of the renilla luciferase gene. Sequences containing a target site are cloned between the two reporter genes such that the downstream firefly luciferase gene is in the same reading frame and the production of full length protein requires reading through the stop codon of the target site. The following p2Luc constructs are created: 0 reading frame is shown:

| | |
|---|---|
| p2Luc-amber | TCG UAG TGG |
| p2Luc-ochre | TCG UAA TGG |
| p2Luc-opal | TCG UGA TGG |

The constructs are transcribed and translated in vitro with antisense oligonucleotides, using Rabbit Reticulocyte lysates in the presence of ³⁵S Methionine and analyzed by electrophoresis on SDS polyacrylamide gels. More specifically, the dual luciferase constructs described above are in some cases added directly to TNT Coupled Reticulocyte Lysate reactions as described (Promega). In other cases, the dual luciferase constructs are linearized with Pml-1 restriction enzyme prior to production of capped mRNA by in vitro transcription reactions utilizing the mMessage mMachine Kit obtained from Ambion, Inc. In the latter case, 0.2 ug of capped mRNA is added to 6.6 ul of Rabbit Reticulocyte Lysate, 70 mM KCL, 0.02 mM each amino acid except Methionine, Tyrosine, and Tryptophan, 0.04 mM Tyrosine and Tryptophan along with 4 uCi of 35S Methionine (1000 Ci/mmol) in a total of 10 ul. The relative abundance of Tyrosine and Tryptophan allow for the efficient recoding of nonsense mutations into codons specifying Tyrosine and Tryptophan. Proteins are separated by SDS polyacrylamide gel electrophoresis and the gels are fixed with 7.5% acetic acid and methanol for 20 minutes. After drying under vacuum, the gels are visualized using a Storm 860 phosphorimager (Molecular Dynamics) and radioactive bands quantified using ImageQuant software.

Percent readthrough is calculated as the percent of full length (readthrough) product relative to the termination product and the full length product combined. The value of each product is corrected for the number of methionine codons present in the coding sequence and stop codon readthrough is identified.

### Example 7. Treatment of an MDX Mouse

The ability of an antisense oligonucleotide to treat a Muscular Dystrophy-like condition in the mdx mouse is determined in vivo in an mdx mouse having a mutation at position 3203 of the mouse dystrophin gene. This mutation, a T to A substitution, results in a premature stop codon (a nonsense mutation) in exon 23. The relevant section of the mouse mdx dystrophin gene is shown below in the 0 reading frame with the nonsense codon underlined:

(SEQ ID NO: 31)
3181-G CAA AGT TCT TTG AAA GAG CAA TAA AAT GGC
TTC AAC TAT CTG AGT GAC ACT GTG.

The following 2′-O-methyl antisense oligonucleotide is designed so as to anneal three nucleotides downstream from the nonsense mutation:

| | |
|---|---|
| CACAGUGUCACUCAGAUAGUUCAAGCC. | (SEQ ID NO: 32) |

Over a four week period, an mdx mouse is given weekly intramuscular injections of 1 μg of the antisense oligonucleotide complexed with 2 μg of Lipofectin (2:1 weight ratio) prepared in saline. See, e.g., Mann et al., 2001. After 4 weeks the injected muscles are analyzed and are found to be producing full length dystrophin protein.

### Example 8. Treating Duchenne Muscular Dystrophy Caused by a Deletion Resulting in Part of the Protein Being Translated in the −1 Reading Frame

The ability of an antisense oligonucleotide to treat Duchenne Muscular Dystrophy (DMD) is determined in vivo in a subject, for example a mammal, having the mutation Id number DMD_e53e60 in the Leiden DMD database available on the world wide web at DMD.n1. This mutation, characterized by the deletion of exons 53 through 60, results in a shift to the −1 reading frame after the translation of exon 52. Downstream of the deletion, in exon 61, a rare codon site (CGA G) exists in the −1 frame. By targeting this site with an antisense oligonucleotide to induce a +1 shift in reading frame, the proper reading frame for the remainder of the protein can be restored. Exon 61, in the −1 frame, has the following mRNA sequence with the rare codon site underlined:

(SEQ ID NO: 33)
GU GGC CGU CGA GGA CCG AGU CAG GCA GCU GCA UGA
AGC CCA CAG GGA CUU UGG UCC AGC AUC UCA GCA CUU
UCU UUC CA.

The following 2′-O-methyl antisense oligonucleotide is designed so as to anneal three nucleotides downstream from the slippery site:

| | |
|---|---|
| GGGCUUCAUGCAGCUGCCUGACUCG. | (SEQ ID NO: 34) |

Over a four week period, the subject is given weekly intramuscular injections of 1 μg of the antisense oligonucleotide complexed with 2 μg of Lipofectin (2:1 weight ratio) prepared in saline. See, e.g., Mann et al., 2001. After 4 weeks the injected muscles are analyzed and are found to be producing full length dystrophin protein minus exons 53 through 60.

### Example 9. Treating Duchenne Muscular Dystrophy Caused by a Deletion Resulting in Part of the Protein Being Translated in the +1 Reading Frame

The ability of an antisense oligonucleotide to treat Duchenne Muscular Dystrophy (DMD) is determined in vivo in a group of subjects having the mutation Id number DMD_e64e65 in the Leiden DMD database available on the world wide web at DMD.n1. This mutation, characterized by the deletion of exons 64 and 65, in a shift to the +1 reading frame after the translation of exon 63. Downstream of the deletion, in exon 66, a slippery site (U UUA AAA) exists in the +1 reading frame. By targeting this slippery site with an antisense oligonucleotide to induce a −1 shift in reading frame, the proper reading frame for the remainder of the protein can be restored. Exons 66 and 67, in the +1 frame, have the following mRNA sequence with the slippery site underlined:

(SEQ ID NO: 35)
GG GAC GAA CAG GGA GGA UCC GUG UCC UGU CUU UUA
AAA CUG GCA UCA UUU CCC UGU ACC UUU UCA AGC AAG
UGG CAA GUU CAA CAG GAU UUU GUG ACC AGC GCA GGC
UGG GCC UCC UUC UGC AUG AUU CUA UCC AAA UUC CAA
GAC AGU UGG GUG AAG UUG CAU CCU UUG GGG GCA GUA
ACA UUG AGC CAA GUG UCC GGA GCU GCU UCC AAU UU.

The following oligonucleotide is designed so as to anneal three nucleotides downstream from the slippery site:

| | |
|---|---|
| UUGAAAAGGUACAGGGAAAUGAUGC. | (SEQ ID NO: 36) |

2′-O-methyl, morpholino, PNA, and phosphorothioate antisense oligonucleotides are produced. Each type of antisense oligonucleotide is tested in a group of subjects. Over a four week period, each subject is given weekly intramuscular injections of 1 μg of a single antisense oligonucleotide complexed with 2 μg of Lipofectin (2:1 weight ratio) prepared in saline. See, e.g., Mann et al., 2001. The DMD is ameliorated in the muscles injected due to the production of full length dystrophin protein minus exons 64 and 65.

All references, including publications, patents, patent applications, mutation Id number, and GenBank Accession Numbers cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

Alff-Steinberger C. (1987). Codon usage in Homon Sapiens: evidence for a coding pattern on the non-coding strand and evolutionary implication of dinucleotide discrimination. J. Theor. Biol. 124, 89-95.
Antonarakis S., M. Krawczak and D. Cooper (2000). Disease-causing mutations in the human genome. Eur. J. Pediatr. 159 (Suppl. 3), S173-S178.
Brierley I., P. Digard and S. Inglis (1989). Characterization of an efficient coronavirus ribosomal frameshifting signal: requirement for an RNA pseudoknot. Cell 57:537-547.
Current Protocols in Molecular Biology (Fred M. Ausubel et al. eds. 1998).
Dunckely M., M. Manoharan, P. Villiet, I. Eperon and G. Dickson (1998). Modification of splicing in the dystrophin gene in cultured mdx muscle cells by antisense oligoribonucleotides. Hum. Mol. Gen. 7 (7) 1083-1090.
Gilis D., S. Massar, N. Cerf and M. Rooman (2001). Optimality of the genetic code with respect to protein stability and amino-acid frequencies. Genome Bio. 2 (11) 1-12.
Goodman and Gilman's The Pharmacologic Basis of Therapeutics (Louis S. Goodman et al. eds., 10th ed. 2001).
Grentzmann G., J. Ingram, P. Kelly, R. Gesteland and J. Atkins (1998). A dual-luciferase reporter system for studying recoding signals. RNA 4:479-486.
Ivaylo P., R. Gesteland, S. Matsufuji and J. Atkins (1998). Programmed frameshifting in the synthesis of mammalian antizyme is +1 in mammals, predominantly +1 in fission yeast, but −2 in budding yeast. RNA 4: 1230-1238.
Kollmus H., A. Honigman, A. Panet and H. Hauser (1994). The sequences of and distance between two cis-acting signals determine the efficiency of ribosomal frameshifting in human immunodeficiency virus type 1 and human T-cell leukemia virus type II in vivo. J. Virol. 68:6087-6091.
Leiden DMD Database, at http://www.DMD.n1 (Jul. 21, 2004).
Mann C., K. Honeyman, A. Cheng, T. Ly, F. Lloyd, S. Fletcher, J. Morgan, T. Partridge and S. Wilton (2001). Anitsense-induced exon skipping and synthesis of dystrophin in the mdx mouse. Proc. Nat. Acad. Sci. 98 (1) 42-47.
Remington's Pharmaceutical Sciences (Alfonso R. Gennaro et al. eds., 18th ed. 1990).
Sharp P., E. Cowe, D. Higgins, D. Shields, K. Wolfe and F. Wright (1988). Codon usage pattern in E. coli, B. subtilis, S. cerevisiae, S. pombe, D. melanogaster and H. sapiens; a review of the considerable within-species diversity. 16 (17) 8207-8211.
Taylor M., J. Paulauskis, D. Weller and L. Kobzik (1996). In vitro efficacy of morpholino-modified antisense oligomers directed against tumor necrosis factor-alpha mRNA. J. Biol. Chem. 271 (29) 17445-17452.
Weiss R., W. Huang and D. Dunn (1990). A nascent peptide is required for ribosomal bypass of the coding gap in bacteriophage T4 gene 50. Cell 62 (1) 117-26.

1. A method of modulating translation comprising:
providing an mRNA molecule that is capable of being translated via a ribosome;
providing an antisense oligonucleotide;
annealing said antisense oligonucleotide to said mRNA molecule; and
translating said mRNA molecule wherein the antisense oligonucleotide produces a modulation of translation at a target site.

2. The method according to claim 1, wherein the antisense oligonucleotide is selected from the group consisting of morpholino, 2′-O-methyl, PNA, RNA, phosphorothioate oligonucleotides, and combinations thereof.

3. The method according to claim 1, wherein said antisense oligonucleotide is between 5 and 100 nucleotides in length.

4. The method according to claim 1, wherein annealing said antisense oligonucleotide is between 10 and 50 nucleotides in length.

5. The method according to claim 1, wherein said antisense oligonucleotide is about 25 nucleotides in length.

6. The method according to claim 1, wherein said antisense oligonucleotide has a A/U:G/C ratio of about 1:1.

7. The method according to claim 1, wherein said annealing occurs from about −5 to about +15 from said target site.

8. The method according to claim 1, wherein said annealing occurs from about −1 to about +7 from said target site.

9. The method according to claim 1, wherein said modulation of translation results in a −1 frame-shift.

10. The method according to claim 1, wherein said modulation of translation results in a +1 frame-shift.

11. The method according to claim 1, wherein said modulation of translation results in a stop codon readthrough.

12. The method according to claim 1, wherein said annealing does not alter splicing.

13. The method according to claim 1, wherein said annealing does not prevent a protein from binding to said mRNA molecule.

14. The method according to claim 1, wherein said target site is a rare codon site.

15. The method according to claim 1, wherein said target site is a slippery site.

16. The method according to claim 1, wherein mRNA is derived from a virus.

17. The method according to claim 1, further comprising providing a cell and introducing said antisense oligonucleotide into said cell.

18. The method according to claim 17, further comprising culturing said cell.

19. The method according to claim 17, wherein introducing said antisense oligonucleotide into said cell further comprises administering said antisense oligonucleotide to a mammal.

20. The method according to claim 19, wherein said mammal is a human.

21. The method according to claim 1, wherein said modulation of translation results in (3N+1) or (3N+2) nucleotides being translated twice, wherein N equals any whole number.

22. The method according to claim 1, wherein said modulation of translation results in (3N+1) or (3N+2) nucleotides not being translated, wherein N equals any whole number.

23. A method of modulating frame-shifting comprising:
providing an RNA molecule that is capable of being translated via a ribosome;
choosing a target site on said RNA molecule;
annealing an antisense oligonucleotide 3′ of a target site; and
translating said RNA wherein a modulation of translation occurs.

24. The method according to claim 23 wherein the target site comprises a slippery site.

25. The method according to claim 24, wherein the nucleotide sequence is not AAAAAAA or UUUUUUU.

26. A method of increasing frame-shifting comprising:
providing an RNA molecule that is translated via a ribosome;
choosing a target site on said RNA molecule;
annealing an antisense oligonucleotide 3′ to the target site; and
translating said RNA molecule, wherein said antisense oligonucleotide increases frame-shifting during translation of said RNA molecule.

27. The method according to claim 26 wherein said annealing does not alter the secondary structure of the RNA.

28. A method of decreasing frame-shifting comprising:
providing an RNA molecule that is translated via a ribosome;
choosing a target site on said RNA molecule;
annealing an antisense oligonucleotide 3′ to the target site; and
translating said RNA molecule, wherein said antisense oligonucleotide decreases frame-shifting during translation of said RNA molecule.

29. A method of increasing stop codon readthrough comprising:
providing an RNA molecule that is translated via a ribosome;
identifying a stop codon on said RNA molecule;
annealing an antisense oligonucleotide 3′ to the stop codon; and
translating said RNA molecule, wherein said antisense oligonucleotide increases stop codon readthrough during translation of said RNA molecule.

30. A method of treating a subject with a disease resulting from a frame-shift mutation in an mRNA, said method comprising:
identifying the site of the frame-shift mutation in said mRNA;
identifying a target site on said mRNA;
creating an antisense oligonucleotide that is capable of annealing 3′ of the target site on the mRNA; and
providing said antisense oligonucleotide to the subject; and
inducing a compensating frameshift in the translation of the mRNA, thereby treating the subject.

31. The method according to claim 30, wherein the subject is a mammal.

32. The method according to claim 31, wherein providing said antisense oligonucleotide to the mammal comprises, administering a pharmaceutical composition to the mammal.

33. The method according to claim 30, wherein the antisense oligonucleotide is selected from the group consisting of morpholino, PNA, RNA, phosphorothioate oligonucleotides, and combinations thereof.

34. The method according to claim 30, further comprising:
identifying a target site 3′ of the frame shift mutation, said target site comprising a slippery site.

35. The method according to claim 34, wherein the nucleotide sequence is not AAAAAAA or UUUUUUU.

36. A medicament comprising:
an antisense oligonucleotide capable of modulating translation; and
a pharmaceutically acceptable carrier.

37. The medicament according to claim 36, wherein the antisense oligonucleotide comprises a sequence capable of annealing to a target site, wherein the target site comprises a slippery site.

38. A method for the treatment of a disorder caused by a frameshift or nonsense mutation in a subject, the method comprising:
administering to the subject an effective amount of an antisense oligonucleotide;
wherein administering to the subject an effective amount of an antisense oligonucleotide treats a disorder caused by a frameshift or nonsense mutation.

39. (canceled)

40. The method of claim 38, wherein the antisense oligonucleotide is selected from the group consisting of morpholino, PNA, RNA, phosphorothioate oligonucleotides, and combinations thereof.

41. The method of claim 38, wherein the disorder caused by a frameshift or nonsense mutation is selecting from the group consisting of Muscular Dystrophy, Ataxia telangiectasia, Cystic fibrosis, Hurler's syndrome, Hypercholesterolemia, Colorectal Adenomatous Polyposis, Insulin-dependent diabetes mellitus, Walker-warburg syndrome, Alstrom syndrome, Wilson disease, and Werner syndrome.

42. The method of claim 38, wherein the disorder is found in a mammal.

43. The method of claim 38, wherein treating the disorder comprises administering a pharmaceutical composition.
